(19) United States
(12) Patent Application Publication
(10) Pub. No.: US 2019/0247606 A1
(43) Pub. Date: Aug. 15, 2019

(54) Variable Temperature Electronic Vaporization Device with Filter Addition and Methods

(71) Applicant: Samuel Alexander Williams, Orlando, FL (US)

(72) Inventor: Samuel Alexander Williams, Orlando, FL (US)

(73) Assignee: Samuel Alexander Williams, Orlando, FL (US)

(21) Appl. No.: 16/392,618

(22) Filed: Apr. 24, 2019

Related U.S. Application Data
(60) Provisional application No. 62/662,199, filed on Apr. 24, 2018.

(51) Int. Cl. A61M 16/10; A24F 47/00; A61M 15/00

(52) U.S. Cl. CPC A61M 16/107; A24F 47/008; F17C 9/02; A61M 15/0013; A61M 15/0086

(57) ABSTRACT

A vaporizer configured in such a way that integrates a user removable and replaceable filtering system in which vapor, aerosols, smoke, and or odors are reduced or eliminated upon use of the filtering system should the user wish to use it. The vaporizer may also integrate in some forms a valve system which restricts or eliminates the flow of air into or out of the device in order to mitigate and or eliminate the escape of smoke, aerosols, vapor, and or odor; of which a user may activate via electrical or mechanical means. The vaporizer integrates logic and control circuitry which controls the power displaced into an integrated or removable heating chamber, atomizer, cartomizer, cartridge, clearomizer and or any other such container in which materials or substances are vaporized or combusted. The logic and control circuitry also employs user input through various means, such as a electromechanical switches and or an accelerometer. The logic and control circuit also provides user feedback via light, sound, and or tactile feedback.

### CROSS-REFERENCE TO RELATED APPLICATIONS

This application claims benefit of the U.S. Provisional Patent Application No. 62/662,199, filed Apr. 24, 2018, the disclosure of which is hereby incorporated herein in its entirety by reference.

### STATEMENT REGARDING FEDERALLY SPONSORED RESEARCH OR DEVELOPMENT

Not Applicable

### REFERENCE TO A SEQUENCE LISTING, A TABLE, OR A COMPUTER PROGRAM LISTING COMPACT DISC APPENDIX

Not Applicable

### BACKGROUND OF INVENTION

Smokers are subject to many health risks associated with the combustion and consumption of combustible and or vaporizable substances and or materials. Smokers subject themselves, their surroundings, and others to the unpleasant, and potentially harmful odors that result from the combustion and consumption of combustible and or vaporizable substances and or materials. The variable temperature electronic vaporization device with filter addition and methods was developed with the purpose of making smoking a healthier, more pleasant experience for users, their environments, and those around them.

### SUMMARY OF INVENTION

Embodiments of the invention are defined by the claims below, not this summary. A high-level overview of various aspects of the invention is provided here to introduce a selection of concepts that are further described in the Detailed-Description section below. This summary is not intended to identify key features or essential features of the claimed subject matter, nor is it intended to be used in isolation to determine the scope of the claimed subject matter. In brief, this disclosure describes, among other things, an electronic smoking device that incorporates a way to conceal and or eliminate any emitted vapor, smoke, and or odor.

The variable temperature electronic vaporization device with filter addition and methods utilizes a chamber in which vaporizable materials and or substances are placed and heated by an element controlled by an electrical circuit in order to produce a vapor that garners a tactile response from the user's mouth and lungs, without in some embodiments causing combustion; unless combustion is desired, of the vaporizable materials and or substances; mitigating health risks associated with the combustion and consumption of vaporizable materials and or substances. In order to mitigate the inconvenience and potentially harmful effects of smoke odor on the user, their environment, and other people nearby, the device has an aerosol percolator assembly add on (also referred to as an filtering add on), through which users expel the smoke, vapor and or aerosol, in turn eliminating any traces of smoke, vapor, and or odor. In addition to the aerosol percolator assembly; the device in some embodiments features a valve system in which the user may activate via electrical or mechanical or combination of the two (electromechanical) means that mitigates and or eliminates the escape of smoke, vapor, aerosol, and or odor to or from the device. The variable temperature electronic vaporization device with aerosol percolator assembly addition also includes a built-in power source so that users may recharge their device for repeated use. The aerosol percolator assembly addition is implicated in such a way that the user is not required to utilize it, should they not wish to do so. The aerosol percolator assembly addition is modular and can be replaced by the user.

### BRIEF DESCRIPTION OF THE DRAWINGS

A better understanding of the features and advantages of the present invention will be obtained by reference to the following detailed description that sets forth an illustrative embodiment, in which the principles of the invention are used, and the accompanying drawings of which:

FIG. 1 Illustrates an embodiment of the invention comprised of a body, single button interface, aerosol percolator, mouthpiece, and user input area which may mechanically or electrically or a combination of the two (electromechanically) activate a valve system.

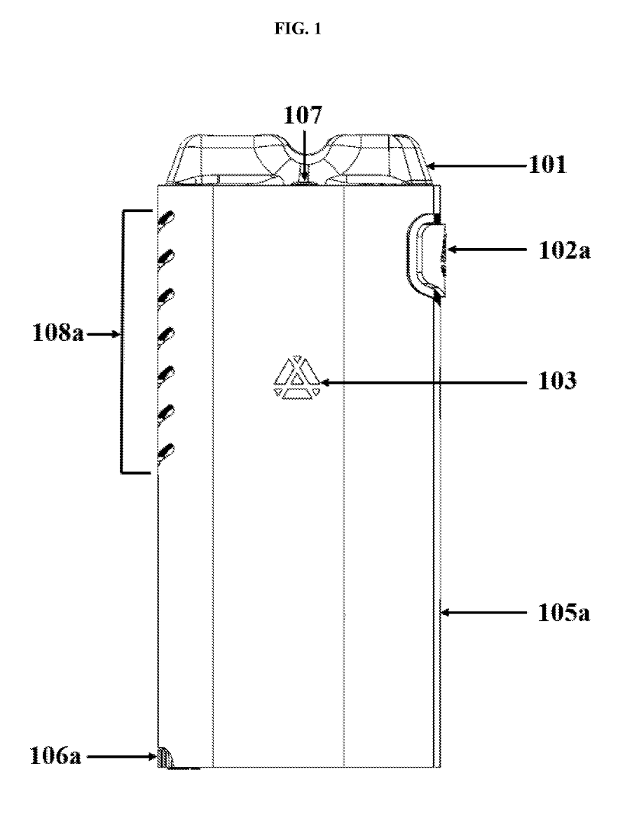

FIG. 2 Is a sectional view of the embodiment in FIG. 1; illustrating the locations of the aerosol percolation assembly, the valve assembly, the power source, and vaporization chamber.

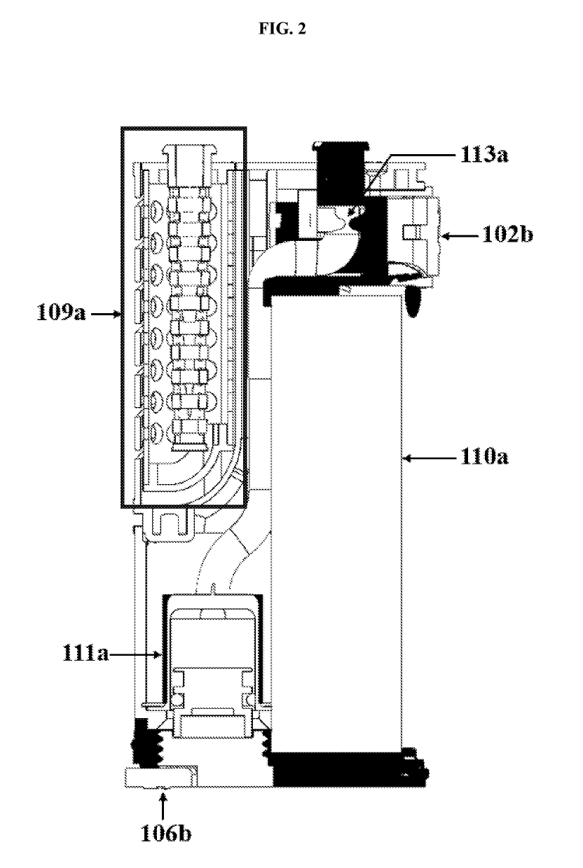

FIG. 3 Is a view of the vaporization chamber present in some embodiments of the device.

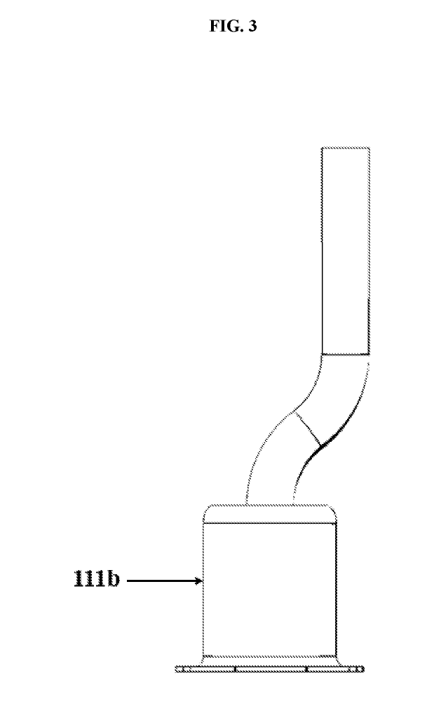

FIG. 4 Is a sectional view of the embodiment in FIG. 1; with an alternate vaporization chamber described below. This view is illustrating the locations of the aerosol percolation assembly, the valve assembly, the power source, and a user removable heating chamber present in some embodiments where materials and or substances are vaporized.

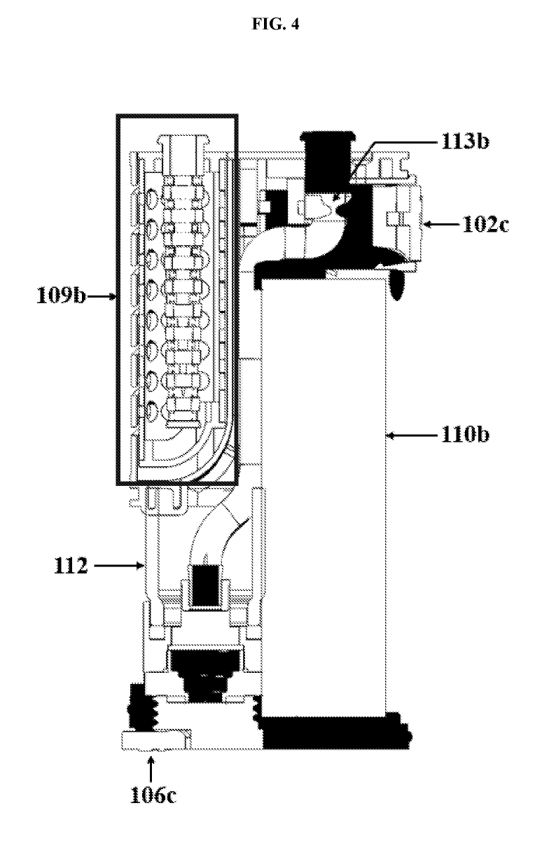

FIG. 5 Is a view of the user removable vaporization chamber as shown in FIG. 4.

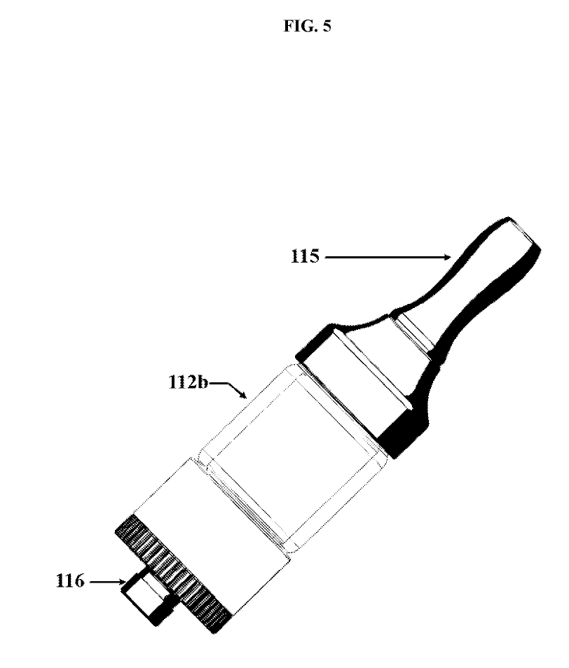

FIG. 6 Is the bottom view of the embodiment in FIG. 1, showing the locations of the charging input, fastening cap, and grip assist tongue.

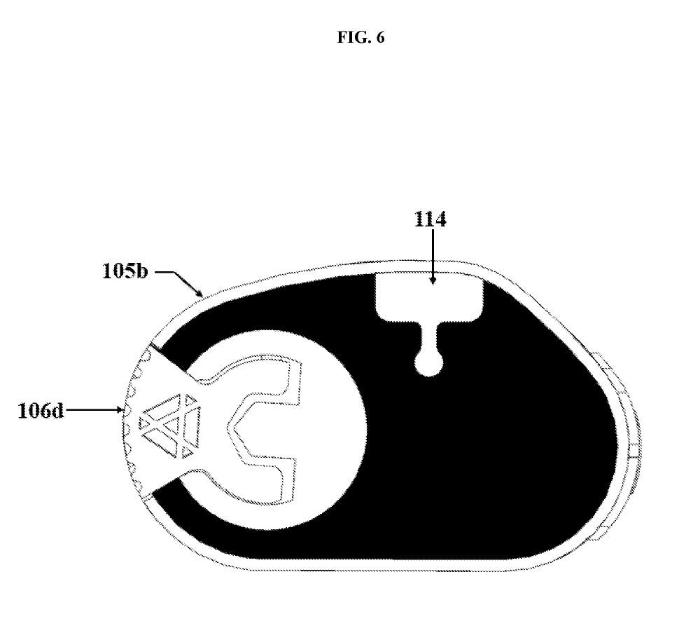

FIG. 7 Is a top view of the embodiment in FIG. 1 with the mouthpieces removed, showing the locations of a user input button, aerosol percolator assembly, valve assembly, and user input area.

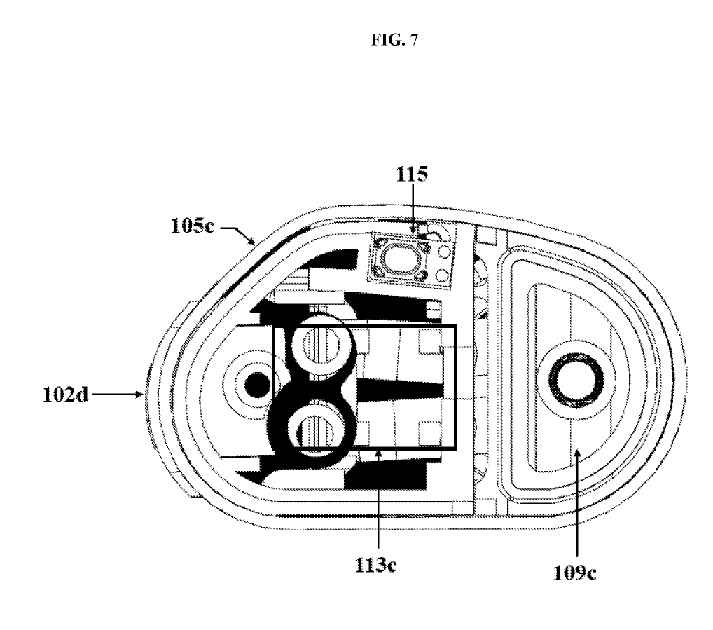

FIG. 8 Is a sectional view of the embodiment in FIG. 1 with the mouthpieces removed, rotated 90 degrees on the Y axis. It further illustrates the locations of the aerosol percolator assembly, vaporization area, power source, fastening cap, and logic and control board.

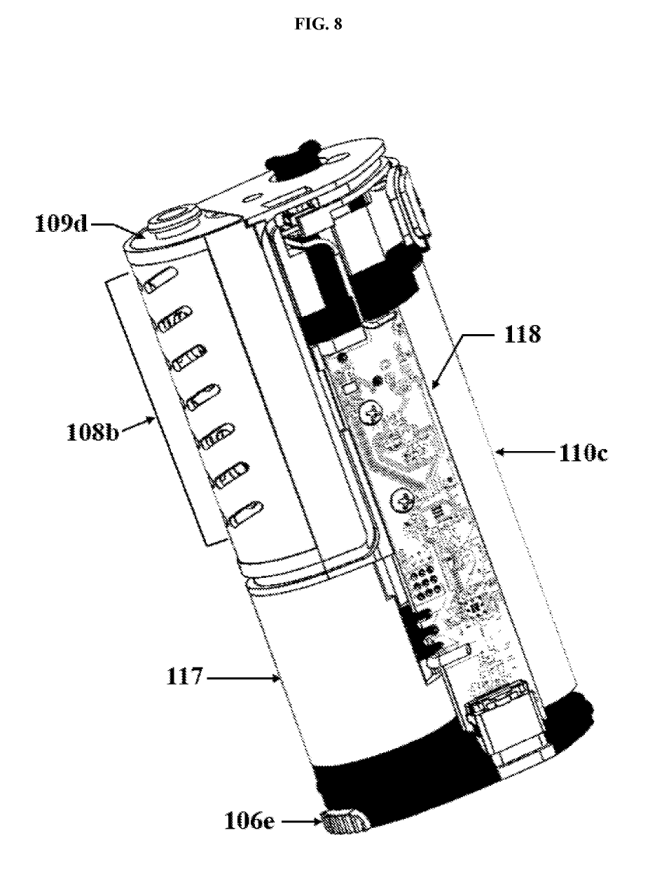

### DETAILED DESCRIPTION OF THE INVENTION

The invention described herein has a wide range of applications for the inhalation of an active substance as will be appreciated by persons of skill in the art upon reviewing the disclosure. The device could be used, for example, to inhale any substance, such as a botanical, pharmaceutical, nutraceutical, oil, wax or any other substance providing a benefit or sensation to an end user.

Provided herein is a device for generating an inhalable aerosol and or vapor comprising of any of the following: a mouthpiece or mouthpieces; a body; an electric heater in some configurations, an oven or container in which materials or substances will vaporize, a printed circuit board within said body, and said electric heater and or heating chamber configured to heat any vaporizable material and or substances to generate an inhalable aerosol or vapor; a temperature sensor; an accelerometer in some configurations; a valve system in some configurations; and an aerosol percolator assembly.

In some embodiments, there is at least one mouthpiece 101 which may or may not be split integrated into the device.

In some embodiments, the heater is adhered to the circuit board 118 via an electrically conductive means. In some embodiments, the electronic heater comprises of a resistive heating element and or temperature sensor configured to monitor and precisely control vaporization temperature of the vaporizable materials and or substances. In some embodiments, the heater circuit is a thin film polyimide heater.

In some embodiments the heating element and or heating chamber may interface with the heater circuit via an electromechanical user removable interface such as but not limited to conductive screw threads, spring loaded pins, tension fit conductive pads 116.

In some embodiments, the device comprises a fastening cap 106a,b,c,d,e with grip assist tongue.

In some embodiments, the device comprises a thermally conductive shell 105a,b,c configured to distribute excess heat and configured to maintain a low exposed surface temperature.

In some embodiments, the device comprises time based, and or sensor based, standby mode activation, and or deactivation of said standby mode. In some embodiments, the sensor comprises an accelerometer or other tactile/vibration sensor.

In some embodiments, the device integrates a proportional integral derivative (PID) control loop configured to control operating temperature.

In some embodiments, the device comprises a thin wall metal heating chamber 111a,b.

In some embodiments, the device may have a removable vaporization and or heating chamber 112a,b in which a heating element and material and or substance is present to be vaporized, smoked, and or aerosolized.

In some embodiments, the device may have a removable vaporization and or heating chamber 112a,b and the user may utilize the mouthpiece of such chamber 115 to inhale vaporized, combusted, and or aerosolized materials or substances.

In some embodiments, the device comprises aerogel insulation or other insulating material. In some embodiments, the aerogel insulation comprises a silica aerogel with reinforcing fibers.

In some embodiments, the device incorporates an input for charging 114.

In some embodiments, the device comprises a single button interface 115.

In some embodiments, the device comprises aerosol percolator assembly 109a,b,c,d.

In some embodiments, the device comprises a user operated valve system 102a,b,c,d.

Provided herein is a device for generating an inhalable aerosol and or vapor comprising of at least one of the following: a body; an electric heater in some configurations, a heating chamber in some configurations, and a printed circuit board within said body, said electric heater configured to heat any vaporizable material and generate an inhalable aerosol and or vapor; a temperature sensor; an accelerometer; a valve system; an aerosol percolator assembly; and a mouthpiece. In some embodiments, there is at least one mouthpiece or mouthpieces that are split and or integrated into the device. In some embodiments, there is at least one mouthpiece purposed for the inhalation of the vapor produced in the vaporization chamber and at least one mouth piece purposed for the exhalation of the inhaled vapor into the aerosol percolator assembly.

Provided herein is a device for generating an inhalable aerosol or vapor comprising: a mouthpiece or mouthpieces; a body; an electric heater in some configurations, a heating chamber in some configurations, and a printed circuit board within said body, said electric heater configured to heat any vaporizable materials or substances to generate an inhalable aerosol or vapor; a temperature regulator; an accelerometer; an aerosol percolator assembly; and a valve system. In some embodiments, the device comprises a user operated electrical or mechanical or a combination of the two, electromechanical, valve actuator system. When engaged, the valve and or valves create an airtight seal between the vaporization chamber and the mouthpiece or mouthpieces, as well as creating an airtight seal between the mouthpiece and or mouthpieces and the aerosol percolator assembly. In some embodiments, the user may hold disengage the valves by interfacing with an electrical or mechanical or combination of the two, electromechanical, user interface area on the body of the device, permitting vapor to be inhaled from, or exhaled into the device. Deactivating said valve system causes one or more valves to return to their static state, closing and recreating the airtight seal.

Provided herein is a device for generating an inhalable aerosol and or vapor comprising: a mouthpiece or mouthpieces; a body; an electric heater, a heating chamber in some configurations, and a printed circuit board within said body, said electric heater configured to heat any vaporizable materials and or substances to generate an inhalable aerosol or vapor; a temperature sensor; an accelerometer; a valve system; and an aerosol percolator assembly.

As described herein, an electronic heater comprises a heater circuit, a heating chamber and a printed circuit board to heat a vaporizable material and or substance to generate an inhalable aerosol. The heater circuit may be flexible in some configurations. In some embodiments, the flexible heater circuits are typically etched from a copper or constantan, clad polyimide film. In some embodiments, a flexible heater is constructed stamping (die-cutting) a thin sheet of constantan or copper. In this case, the heater circuit would have to be electrically insulated from adjacent conductive elements in the assembly, using polyimide or other suitable insulation that is non-conductive. The heater circuit heats the attached chamber which then heats the vaporizable material by thermal conduction, convection and or radiation. The resistive heater circuit heats up as current passes through it. Heat is then conducted from the circuit to the oven walls. Thermal conduction continues from the oven walls into the vaporizable material. Note that heat also transfers from the oven walls into the vaporizable material via convection and or radiation, but most transfer occurs via conduction.

Provided herein is a device for generating an inhalable aerosol and or vapor comprising: a mouthpiece or mouthpieces; a body; an electric heater, a heating chamber in some configurations, and a printed circuit board within said body, said electric heater configured to heat any vaporizable materials and or substances to generate an inhalable aerosol or vapor; a temperature sensor; an accelerometer; a valve system; and an aerosol percolator assembly; and a time or sensor based standby activation to conserve battery power. This may alternatively be called sleep, or sleep mode. After non-use based on time, movement, or lack thereof, position (e.g. vertical, and or horizontal), or after any combination of these, the device is programmed to convert to sleep mode (standby mode), in order to conserve battery power, at least. The device may be awoken from this standby or sleep mode by a change in any of movement (e.g. horizontal from vertical, vertical from horizontal, or movement indicating the user has picked up the device), user touch, the user puffing on the device, or activation by pressing any button on the device (or any combinations thereof). After an extended period in standby mode, the device may turn off, and may be awoken and/or turned on by the user pressing the button on the device, in some embodiments. In such an embodiment, simply moving or removing it from the charger will not activate the device once turned off

In some embodiments, standby mode conserves battery power by lowering the regulation temperature of the device. For example, a large portion of the heat generated by the device is lost to the environment, whether the user is using it or not. However, when the device wakes from standby, it is desirable for it to return to the main operating temperature as quickly as possible, as to give the impression of an uninterrupted experience to the user. For example, if the main operating temperature is 165 degrees Celsius, and standby temperature is 150 degrees Celsius. This temperature difference is slight enough that if the user wakes the device from standby, by the time the user starts using the device, the heater has had enough time to raise the temperature and the user perceives little or no interruption in production of vapor. In some embodiments, the temperature difference is set to be (in degrees Celsius) 30, 25, 20, 15, 10, or 5 degrees between the main operating temperature and standby temperature. In some embodiments, the temperature difference is set to be any temperature from 200 degrees Celsius to 5 degrees Celsius between the main operating temperature and standby temperature.

In some embodiments, the battery is a rechargeable battery 110a,b,c. In certain embodiments, the rechargeable battery is a lead-acid, nickel cadmium (NiCd), nickel metal hydride (NiMH), lithium ion (Li-ion), lithium ion polymer (Li-ion polymer or LiPo), or the like.

A rechargeable battery, storage battery accumulator is a type of electrical battery. It comprises one or more electrochemical cells and is a type of energy accumulator. It is known as a secondary cell because its electrochemical reactions are electrically reversible. Rechargeable batteries come in many different shapes and sizes, ranging from button cells to megawatt systems connected to stabilize an electrical distribution network. Several different combinations of chemicals are commonly used, including lead-acid, nickel cadmium (NiCd)m nickel metal hydride (NiMH), lithium ion (Li-ion), and lithium ion polymer (Li-ion polymer, Li-poly, Li-Pol, LiPo, LIP, PLI, or LiP). In some embodiments the device may contain any of the listed power sources but is not limited to those listed.

The device can create temperatures high enough to aerosolize and or vaporize a product contained within the device. An exemplary device can comprise a mouthpiece and or mouthpieces and a body having a heater in some configurations, a heating chamber in some configurations, a battery, and a controller for maintaining the operating temperature. A user-selected temperature, as described above, could be used as an input to this system. In some embodiments, the temperature could be pre-set. Examples of operating temperature regulators of a device include a bimetallic actuator. Alternatively, a system could be employed to measure the current temperature, for example, with a thermocouple sensor and compare it to a prescribed temperature for example, with a micro-controller. A user-selected temperature, as described above, the selected temperature could be used as an input to this system typically, the operating temperatures of this device are not above 240 degrees Celsius. In some embodiments a microcontroller or via other electronic or mechanical, or combination thereof (electromechanical) actuates a controlling valve, for example, servo, ball valve, pinch valve, solenoid valve, or other valves in which the device would be able to restrict airflow.

Provided herein is a device for generating an inhalable aerosol comprising: a mouthpiece; a body; an electric heater comprising a heater circuit, an oven, and a printed circuit board within said body, said electric heater configured to heat any vaporizable material and generate an inhalable aerosol; a temperature regulator; an accelerometer; a valve system; a aerosol percolator assembly; and a temperature control loop. In certain embodiments provide the heater with soldered thermistor element for the control loop. In certain embodiments, the device comprises a PID (proportional integral derivative) control loop to control operating temperature. The control loop serves to precisely regulate the desired setpoint temperature for the device. Depending on the design and intended use of the device, the set point temperature, in some embodiments, is fixed; in other embodiments, the set point temperature is user-selectable. The set point can also change dynamically during the device operation. For example, in standby mode the setpoint is lowered a certain amount. In some embodiments, the input for the control loop is typically a thermistor, located on or adjacent to the heater circuit. This thermistor leads to a microcontroller which makes A/D measurements and the resulting value is used in calculating the PID control variable. The control variable then sets the duty cycle (and resulting power output) of the heater circuit.

Provided herein is a device for generating an inhalable aerosol and or vapor comprising: a mouthpiece and or mouthpieces; a body; a heating chamber, a printed circuit board within said body, said electric heater configured to heat any vaporizable material and generate an inhalable aerosol and or vapor; an accelerometer; a valve system; an aerosol percolator assembly; and a temperature sensor; wherein the heating chamber circuit has low resistance such that a single battery is capable of powering the device. In some embodiments, the heater circuit resistance is chosen such that the power output of the heater circuit is high enough to reach the desired operating temperature, within an acceptable time period, and such that it can withstand the loading of the system by a user using the device. A rough calculation is provided by the relation: R=V/I, where V is the battery voltage under load, I is the current drawn from the circuit, and R is the heater circuit resistance.

Provided herein is a device for generating an inhalable aerosol and or vapor comprising: a mouthpiece and or mouthpieces; a body; an electric heating chamber which may be referred to as an “oven”, a printed circuit board within said body, said electric heating chamber configured to heat any vaporizable material and generate an inhalable aerosol and or vapor; a temperature sensor; an accelerometer; a valve system; an aerosol percolator assembly. In some embodiments, the oven is thermal pressed (e.g. heat staked or swaged), ultrasonic bonded or over-molded into a high temperature capable plastic component. The processes create a hermetic or dust seal (air tight seal), which prevents environmental dust from entering the internal chambers of the device, as well as any dust from the internal insulation materials from escaping the device and entering the heating chamber. The plastic component may comprise of any thermoplastic materials that provide high temperature stability. In some embodiments, the plastic component comprises polyphenylene sulfide (PPS, trade name Rayton), polyetherimide (PEI, trade name Ultem), liquid crystal polymer (LCP), or the like.

In some embodiments, the oven is heat staked or heat swaged into a high temperature capable plastic component as referring herein, with heat swaging, material is formed all the way around the perimeter of the of the mating edge. With heat staking, there would have a few posts of the thermoplastic that insert through holes in the formed metal oven, and then the posts are heated to form “rivets” of a sort. In certain embodiments, the oven is heat swaged into a high temperature capable plastic component. In some embodiments, the oven is joined to the plastic component by mechanical mechanism, such as using a crimp, threaded connection, press fit, or any of the like. For any mechanical joining, in some embodiments, an o-ring is used between the two components to ensure that a dust seal is created.

Provided herein is a device for generating an inhalable aerosol and or vapor comprising: a mouthpiece and or mouthpieces; a body; an electric heating chamber which may also be referred to as an “oven”, a printed circuit board within said body, said electric heating chamber configured to heat any vaporizable materials and or substances to generate an inhalable aerosol and or vapor; a temperature sensor; an accelerometer; a valve system; an aerosol percolator assembly; and thermally insulating material. In some embodiment, the thermally insulating material is an aerogel blanket, or other fabric that may withstand elevated temperatures. In some embodiments, the device comprises of an insulation chamber that includes thermally insulating material to maintain efficiency and a low exposed surface temperature. In some embodiments, the thermally insulating material may be a silica aerogel with reinforcing fibers (e.g., Pyrogel 2250 flexible aerogel blanket, aerogel, or other insulating materials).

As provided herein, the term “aerogel” refers to a synthetic porous material derived from a gel, in which the liquid component of the gel has been replaced with a gas. The result is a solid with extremely low density and thermal conductivity. Aerogels are good thermal insulators because they almost nullify the three methods of heat transfer (convection, conduction, and radiation). They are good conductive insulators because they are composed almost entirely from a gas, and gasses are very poor heat conductors. Silica aerogel especially good because silica also a poor conductor of heat (a metallic aerogel, on the other hand, would be less effective). They are effective convective inhibitors because air cannot circulate through the lattice. Silica aerogel is the most common type of aerogel and the most extensively studied and used. It is a silica-based substance, derived from silica gel. Carbon aerogels composed of particles with sizes in the nanometer range, covalently bonded together. They have very high porosity (over 50% pore diameter under 100 nm) and surface areas ranging between 400-1000 m2/g. Aerogels made with aluminum oxide are known as alumina aerogels. These aerogels are used as catalysts, especially with “doped” with a metal different from Al. Nickel-alumina aerogel is the most common combination.

Provided herein is a device for generating an inhalable aerosol and or vapor comprising: a mouthpiece and or mouthpieces; a body; an electric heating chamber which may be referred to as an “oven”, a printed circuit board within said body, said electric heating chamber configured to heat any vaporizable material and generate an inhalable aerosol and or vapor; a temperature sensor; an accelerometer; a valve system; and an aerosol percolator assembly contained entirely within the body. In some embodiments, the aerosol percolator assembly can be utilized by exhaling inhaled aerosol or vapor into the aerosol percolator assembly, in some embodiments this may be through a mouthpiece and or mouthpieces. The vapor and or aerosol is pushed through an aerosol percolator assembly, effectively eliminating all visible and or invisible traces of smoke and/or vapor and or aerosol and may also eliminate any odor resulting from the vapor and or aerosol, and is expelled through one or more orifices in the body of the device. In some embodiments, the aerosol percolator assembly is modular, and can be removed by the user. In some embodiments, the aerosol percolator assembly is used via at least one mouthpiece specific to its use.

In some embodiments, the aerosol percolator assembly is comprised of at least one appropriate particulate filtering medium and one appropriate odor eliminating medium, or any combination thereof. Appropriate filtering and odor eliminating mediums include, but are not limited to, large particulate membrane filters, paper filters, HEPA filters (high-efficiency particulate air filter), fiberglass filters, polyester and pleated filters, and activated carbon filters.

Provided herein is a device for generating an inhalable aerosol and or vapor comprising: a mouthpiece and or mouthpieces; a body; an electric heating chamber which may be referred to as an “oven”, a printed circuit board within said body, said electric heating chamber configured to heat any vaporizable material and generate an inhalable aerosol and or vapor; a temperature sensor; an accelerometer; a valve system; an aerosol percolator assembly; and a charging input 114.

In some embodiments, the battery used in the device is of a lithium-ion chemistry for repeated uses of the device. The device can be used for up to 10, 20, 30, 40, 50, 60, or more uses (depending what size of the rechargeable battery is employed). In some embodiments, the device can be used for more than 60 uses. The device can also be used for up to 1, 2, 3, 4, 5, 6, 7, or 8 hours or more of continuous or non-continuous use. The long-lasting use of the device provides the user the advantage of not having to service the device or recharge the battery on a regular basis.

Typically, the operating temperature of the device is no more than 250 degrees Celsius. Often, the temperature required to aerosolize and or vaporize a product is between about 100 to 250 degrees Celsius. Once the product within the device has been aerosolized and or vaporized, the aerosolized and or vaporized product is provided to a user through at least one mouthpiece. In many cases and the device is designed to emulate a smoking device, such a cigarette, a pipe, cigar, or other device in which a user may inhale combustible, and or vaporizable materials or substances.

Provided herein is a device for generating an inhalable aerosol and or vapor comprising: a mouthpiece and or mouthpieces; a body; an electric heating chamber which may be referred to as an “oven”, a printed circuit board within said body, said electric heating chamber configured to heat any vaporizable material and generate an inhalable aerosol and or vapor; a temperature sensor; an accelerometer; a valve system; an aerosol percolator assembly; a button-operated temperature selection, a visual indicator, and in some configurations an audible indicator and/or other sensory output (e.g., vibration). In some embodiments, a tactile (mechanical, electrical, or electromechanical) switch is used as an input to a microcontroller which, via its software, indicates the change to the user (e.g., visual LED, audible, vibration, wireless communication or the like), and changes the set point temperature of the device. The switch can also be capacitive, resistive, or the like.

In some embodiments, the vaporization device comprises a thin wall metal heating chamber (or oven chamber). Thin walls allow for low thermal mass and thus speedy warmup times. The terms “heating chamber”, “oven chamber”, “electronic heater”, “electronic heating chamber”, “electronic vaporization chamber”, and “vaporization chamber” are used interchangeably.

Provided herein is a device for generating an inhalable aerosol and or vapor comprising: a mouthpiece and or mouthpieces; a body; an electric heating chamber which may be referred to as an “oven”, a printed circuit board within said body, said electric heating chamber configured to heat any vaporizable material and generate an inhalable aerosol and or vapor; a temperature sensor; an accelerometer; a valve system; an aerosol percolator assembly; a fastening cap with grip assist tongue configured to cover the vaporization chamber 106a,b,c,d,e. The terms “fastening cap with grip assist tongue”, “fastening cap”, “threaded cap”, and “cap” are used interchangeably. In some embodiments, the fastening cap with grip assist tongue is threaded into the thermo-plastic element surrounding the vaporization chamber. The fastening cap is nominally recessed entirely into the body of the device. This is to prevent inadvertent removal of the fastening cap in the user's pocket, purse, or other means in which it may be stored. To remove the fastening cap, in some embodiments, the user lifts and extends the grip assist tongue on the cap, and unscrews the cap in a counterclockwise and or clockwise fashion, thus removing the cap entirely and allowing access to the vaporization chamber. The cap can be re-fitted onto the device, in some embodiments, by lifting the tongue on the cap, placing the fastening cap over the threads surrounding the vaporization chamber, rotating the cap in a clockwise or counterclockwise fashion, and pressing the tongue into the fastening cap so that it is flush with the cap, in such a fashion as to prevent accidental opening.

One of ordinary skill in the art would readily employ energy supply sources to charge the battery. In some embodiments, the energy supply source is a wall mount charger. In some embodiments, the energy supply source is a car charger. In some embodiments, the energy supply source is a portable charger. In some embodiments, the energy supply source is a USB compatible device such as but not limited to a computer. In certain embodiments, the energy supply sources include solar powered, wind powered or other green energy powered chargers.

In some embodiments, the device comprises a thermally conductive shell to distribute excess heat and maintain low exposed surface temperature. In some embodiments, the thermally conductive shell is made of materials having low specific heat but high thermal conductivity. In some embodiments, the configuration of materials in the thermally conductive shell is such that the temperature of the shell is below 140 degrees F., below 130 degrees F., below 120 degrees F., below 110 degrees F., below 100 degrees F., at or below 140 degrees F., at or below 130 degrees F., at or below 120 degrees F., at or below 110 degrees F., at or below 100 degrees F., at or below 98.6 degrees F., at or below 90 degrees F., at or about room temperature, at or below about 140 degrees F., at or below about 130 degrees F., at or below about 120 degrees F., at or below about 110 degrees F., at or below about 100 degrees F., or at or below a temperature at which skin will burn after 2 seconds of touch, at or below a temperature at which skin will burn after 5 seconds of touch, at or below a temperature at which skin will burn after 10 seconds of touch, and/or about at room temperature. This combination means that heat will spread quickly, but when held there is not much energy to be absorbed into the users physical retention means. In some embodiments, the thermally conductive shell is made of aluminum, or the like In some embodiments, the energy supply source is a portable charger; an aerosol percolator assembly; and a thermally conductive shell configured to distribute excess heat and maintain a low exposed surface temperature.

Any material that is capable of being aerosolized and or vaporized and inhaled by a user may be incorporated into the device of the invention as would be obvious to one skilled in the art. It is of particular interest that the material provides an experience to the user either in terms of tactile response in the respiratory tract, or in terms of visual feedback regarding the exhalation of the inhaled material. For example, many materials have been contemplated for use with the present invention, including, but not limited to, those containing tobacco, natural or artificial flavorings, coffee grounds or coffee beans, mint, chamomile, tea leaves, cocoa, and other non-tobacco alternatives based on other botanicals.

Active elements contained in botanicals vaporize at various temperatures. The device can be calibrated to establish a single stable temperature, intended for aerosolizing and or vaporizing various products; discussed above. A controller can also be used to select a variety of temperature settings.

The user is prevented from touching the hot internal elements by surrounding insulating features. An exemplary device can include insulation for keeping the user from contacting the potentially elevated temperature portion of the device. While greater thermal insulating ability is preferable so that the device performs with the best efficiency possible, an important aspect for the user is to perceive a relatively cool surface temperature. Various strategies can be employed to address the perception of the user regarding the temperature of the device. The device may be wrapped in a thermal insulating material that has enough durability for external use. Materials for this purpose have low thermal conductivity and low thermal capacity (specific heat). The combination of these properties can allow little heat to be transferred to the physical retention means of the user. Examples of materials with low thermal conductivity and capacity include some polymers and ceramics. A separate strategy is to use standoff features that keep the user from coming into contact with the higher temperature area directly. This can also minimize the contact area of the user's physical retention means of the device to additionally reduce perceived heat. The thermal conductivity and specific heat of the standoff features should be as low as possible.

Although preferred embodiments of the present invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein can be employed in practicing the invention and that methods and structures within the scope of these claims and their equivalents be covered thereby.

1. A variable temperature electronic vaporization device, comprising:
a. A filter in which smoke, vapor, and or odor is mitigated or eliminated within.
b. In some embodiments a valve system which mitigates the release of any smoke, vapor, and or odor from said device.
c. Logic and control circuitry operable to maintain user specified temperatures of a heating control circuit, heater, temperature sensing, provide user feedback, control charging and discharging of an integrated power source.
d. A rechargeable battery positioned within the device itself, in which the logic and control circuit makes use of
e. An affixed or removable or any combination thereof heating apparatus, oven, atomizer, cartomizer, cartridge, and or clearomizer, in which materials or substances may be aerosolized, vaporized, and or in some embodiments combusted.

2. The variable temperature electronic vaporization device of claim 1, wherein one may make use of the filter in such a way that it eliminates any smoke, vapor, or odor produced from the device.

3. The variable temperature electronic vaporization device of claim 1, wherein some embodiments may include a valve system with which one would engage or disengage to allow or disallow for the free movement of smoke, vapor, and or odor to or from the device.

4. The variable temperature electronic vaporization device of claim 1, wherein the logic and control circuitry maintains a user specified temperature of an internal or external heating apparatus.

5. The variable temperature electronic vaporization device of claim 1, wherein the logic and control circuitry makes use of a temperature sensor to control heating circuitry.

6. The variable temperature electronic vaporization device of claim 1, wherein the logic and control circuitry implements user feedback via means of light, sound, and or tactile response.

7. The variable temperature electronic vaporization device of claim 1, wherein the logic and control circuitry controls the rate of charge and or discharge of an rechargeable battery.

8. The variable temperature electronic vaporization device of claim 1, wherein some embodiments may make use of an integrated and or removable heating apparatus, oven, atomizer, cartomizer, cartridge, and or clearomizer in which a material or substance is aerosolized, vaporized, and or in some embodiments combusted; which may be delivered to the end user via any physical means such as oral, nasal, and or tactile transfer to garner a tactile response from the user's mouth and or lungs.

9. The method of claim 3, wherein a valve system may incorporate, but not limited to an external peristaltic pinch system, ball valve, gate valve, butterfly valve, check valve, plug valve, globe valve, diaphragm valve, pinch valve, and or needle valve to reduce and or stop airflow into or out of the device as a way to mitigate or eliminate the release of smoke, aerosols, vapor, and or odor.
